(19) United States
(12) Patent Application Publication
(10) Pub. No.: US 2026/0345150 A1
(43) Pub. Date: Nov. 5, 2026

(54) SURGICAL SUTURE SYSTEM COMPRISING AN IMPROVED CAP

(71) Applicant: Fondation de Cooperation Scientifique - FCS IHU de Strasbourg, Strasbourg (FR)

(72) Inventors: Marie-Muguet Klein, Lutzelhouse (FR); Lee Swanström, Portland, OR (US)

(21) Appl. No.: 19/102,717

(22) PCT Filed: Aug. 11, 2023
(86) PCT No.: PCT/EP2023/072282

§ 371 Date: Feb. 10, 2025

(30) Foreign Application Priority Data
Aug. 11, 2022 (FR) FR2208251

(51) Int. Cl. A61B 17/04; A61B 17/00; A61B 17/06

(52) U.S. Cl. CPC A61B 17/0469; A61B 17/0482; A61B 17/06066; A61B 2017/0034; A61B 2017/0608

(57) ABSTRACT

A surgical suture system includes an endoscopic cap, a needle, and a tool for gripping the needle that is axially movable relative to the cap. The needle is temporarily retained by means for holding the needle in the cap, when the gripping tool is in the rear position in a position suitable for gripping by the gripping tool. The cap additionally has a means for holding a suture thread fitted on the needle.

### CROSS-REFERENCE TO RELATED APPLICATIONS

This application is a national phase entry under 35 U.S.C. § 371 of International Patent Application PCT/EP2023/072282, filed Aug. 11, 2023, designating the United States of America and published as International Patent Publication WO 2024/033520 A1 on Feb. 15, 2024, which claims the benefit under Article 8 of the Patent Cooperation Treaty of French Patent Application Serial No. FR2208251, filed Aug. 11, 2022.

### TECHNICAL FIELD

The present disclosure relates to the field of medical devices for endoscopic surgical suturing, and more particularly suturing by flexible endoscopy.

### BACKGROUND

Endoscopic surgery is a method of visually exploring the inside of a cavity, using tubular optical equipment introduced either through the body's natural orifices (flexible endoscopy), or through incisions a few millimeters deep in the skin (laparoscopy). This method enables the insertion and use of instruments within the body to perform surgical operations. This type of minimally invasive surgery offers many advantages over conventional surgical techniques (reduced post-operative pain, recovery time, complications, etc.).

Numerous miniaturized instruments have been developed for this purpose, including forceps, scissors, electric scalpels and more. All these miniaturized instruments are designed to be introduced into the body either via small-diameter trocars (laparoscopy), or through the operating channel of flexible endoscopes.

In the specific field of endoscopic wound closure, surgeons have a variety of techniques at their disposal. These techniques involve either stapling or clipping or suturing using a remote-controlled needle and suture thread. The present disclosure belongs to the latter family of solutions.

In the field of conventional (open) surgery, numerous developments are focusing on specific needles with optimized tapered tips, a curved shape for more precise rotation, and use of a needle holder to easily grasp the needle, irrespective of the user's lateral position. With the evolution of practices and the advent of laparoscopy, the problem of the safe introduction of the needle into the patient has arisen. Today, the trocars used to introduce laparoscopic tools have holes large enough to allow the passage of these standard needles, and several technical solutions have been developed to facilitate the delivery and handling of the needle.

However, when it comes to flexible endoscopy, the problem remains unresolved. Since the operating channel of the endoscope offers only very narrow access, it does not allow the curved needle to pass inside. The technical problem is to bring the curved needle outside the flexible endoscope without damaging the walls of the gastrointestinal tract. The present disclosure relates more particularly to the family of curved needles adapted to new techniques for suturing in flexible endoscopy.

In the state of the art, Patent Application Publication No. US2022225983 is known, which discloses a camera needle adapter that comprises a tube-shaped body having a proximal end, a distal end, a longitudinal axis and a lumen extending along the longitudinal axis. A centrally located side access opening is formed in an outer wall of the tube-shaped body. A needle attachment channel is formed in the outer wall and extends between the side access opening and the distal end of the tube-shaped body. A curved suture needle is positioned inside the needle attachment channel. The needle attachment channel and the curved suture needle extend along an axis that defines an oblique angle with the longitudinal axis of the camera needle adapter. A distally-facing viewing device is positioned inside the lumen at the proximal end of the tube-shaped body. The side access opening, needle attachment channel and curved suture needle are located in a field of view of the viewing device.

Patent Application Publication No. US2020390436 relates generally to an apparatus, devices and methods for increasing force distribution at a tissue-suture interface. In some embodiments, a tissue suturing device may comprise a deployment device and a plurality of tissue supports deployable from the deployment device to a target tissue. The plurality of tissue supports can each comprise a central opening for receiving a suture during deployment.

The prior art solutions take up considerable space, partially masking the outlet of the operator channel wherein the needle actuating mechanism (or any other endoscopic tools that may be required for the surgical procedure) is positioned, and also limiting the field of view of the endoscopic camera.

Additionally, their use is relatively complex and unusual for the surgeon. Firstly, the suture is made frontally, rather than tangentially as in open or laparoscopic surgery. Secondly, the needle is not free to move, but attached to the endoscope or device. Finally, prior art solutions require great dexterity on the part of the operator to grasp the needle, which is located at a distance from the operator, and then position and move it to the area to be sutured, without obscuring vision or damaging the walls of the gastrointestinal tract.

### BRIEF SUMMARY

The needles used in open or laparoscopic surgery have already proved their effectiveness, and should be able to be used in flexible endoscopy. Unfortunately, the main problem is managing the needle, which must be able to be brought in, delivered and removed safely despite its large size and curved shape. The introduction and withdrawal of the needle must not cause any damage to the tissues of the digestive tract. At this time, there is no satisfactory solution.

Thus, the present disclosure, an example of which will be disclosed in greater detail as a non-limiting example, relates in its most general sense to a surgical suturing system enabling suturing to be carried out in endoscopy, and more particularly in flexible endoscopy, using standard and known tools in order to reduce purchase costs, increase ease of use and obtain constant visibility over the procedure. Thus, the use of an endoscopic needle holder and a standard surgical needle, in conjunction with a conventional endoscope, would meet these criteria enabling tangential suturing to be performed using a free needle controllable in different spatial directions, namely translation and rotation.

The purpose of the present disclosure is to provide an endoscopic cap:
a) enabling a large, curved, standard surgical needle to be delivered and withdrawn safely and atraumatically from the gastrointestinal tract,
b) enabling the needle to be handled and delivered/retrieved in a controlled, progressive manner using an endoscopic needle holder, and
c) not restricting the user's visibility.

An endoscopic cap is an accessory generally composed of two parts:
a distal part made of hard or soft transparent plastic in contact with the walls of the digestive tract, and
a proximal part made of transparent soft plastic for attachment to the distal end of a flexible endoscope.

This accessory defines the operating field at the end of the endoscope, which features an operator channel for the passage of a tool for gripping the needle. It allows the tissue to be vacuumed if necessary and treated with various tools. This type of cap is generally non-functionalized.

It is intended to be fitted to the distal end of a conventional flexible endoscope to complement a tool for gripping the needle such as an endoscopic needle gripping tool of the type disclosed in patent application FR3055793, to form a complete endoscopic suturing system.

In order to meet these three essential functions, the present disclosure relates to a surgical suturing system comprising an endoscopic cap, a needle and a tool for gripping the needle, the end of which is axially movable in translation and rotation relative to the cap, ensuring that the needle is held in a position allowing it to be gripped by the gripping tool, for example, a needle holder, extracted from the cap by axial displacement of the gripping means, and then the needle extracted from the cap is manipulated, wherein:
the needle is retained in the cap when the gripping tool is in the rear position,
the needle is temporarily retained by two diametrically opposed holding zones of the inner surface of the tubular wall of the endoscopic cap and retains a constant shape irrespective of temperature, unlike a shape memory needle,
the needle is pushed back by the gripping tool when the gripping tool is actuated into a position suitable for gripping the needle, and
the cap further features means for holding the suture thread fitted on the needle.

The needle is not flexible or shape-memory, but of the “rigid elastic” type, that is, made of a material that deforms slightly and reversibly when subjected to stress on the two arcuate ends of the needle, in the direction of the needle's chord, and returns to its initial shape at any temperature when the stress ceases. Typically, this is a material such as steel or a steel alloy.

The endoscopic cap is designed to contain a needle and hold it in place, in particular, a standard curved surgical needle, while protecting the sharp ends of the needle:
a means of temporarily holding the needle can be formed by one or more slots inside the cap, enabling the needle to be placed and held,
a suture thread-holding means connected to the needle can be formed by one or more grooves inside or outside the cap to hold the suture, or by a biocompatible adhesive,
customized dimensions adapted to suture dimensions, and
the needle is positioned in the cap to protect the sharp ends of the needle and facilitate gripping.

Apart from injury to the gastrointestinal wall by the needle during insertion and removal, another major risk with endoscopic suturing is losing the needle. Indeed, if the free needle were dropped into the stomach or colon, the peristalsis of these organs would make the probability of finding it low. A needle lost in this way poses a serious danger to the patient as it passes through the digestive tract. The fear of losing a needle is one of the reasons why current suture solutions are mostly attached to the end of the endoscope. The unique solution of this disclosure requires the needle to be grasped by the endoscopic needle holder prior to deployment, as needle friction in the endoscopic cap requires intentional force to deploy it, and above all, controlled progressive deployment of the suture by the endoscopist (safety and efficiency). It is only these mechanisms that enable the endoscopist to perform essential tasks such as releasing the needle and re-engaging it in another way to enable effective suturing without the risk of losing the needle.

The endoscopic cap is designed so that the position of the needle makes it accessible to the endoscopic needle holder, thus allowing gradual release of the needle owing to:
the generation of sufficient resistance to hold the needle (no spontaneous release) but allowing active release using an endoscopic needle holder,
the generation of sufficient resistance to hold the suture thread (no spontaneous release) but allowing active release using an endoscopic needle holder, and
this endoscopic cap is designed to allow the needle to be positioned without significantly impeding visibility.

The present disclosure relates, in particular, to a surgical suture system including an endoscopic cap with a tubular wall, a needle and a tool for gripping the needle that is axially movable with respect to the cap. The gripping tool is able to be inserted into the lumen of an endoscope and to slide axially on an axis parallel with the longitudinal axis of the cap. The needle is retained—when the gripping tool is in the rear position—by friction within the two holding zones. The opposite ends of the needle are in a position suitable for gripping by the gripping tool. The cap further features means for holding a suture thread fitted on the needle.

The gripping tool may, for example, be an endoscopic needle holder of the type disclosed in patent EP3509503.

Preferably, the needle is arcuate.

Advantageously, the suture thread holding means can be formed by a housing.

According to a variant, at least one of the needle holding zones is constituted by a longitudinal slot provided on the tubular wall of the endoscopic cap.

According to another variant, at least one of the needle holding zones is constituted by two protruding slots defining a longitudinal guide slot for the ends of the needle.

According to another variant, the tubular wall of the cap has two diametrically opposed holding zones.

Advantageously, the means for holding the suture thread fitted on the needle is constituted by a peripheral groove formed in the tubular wall of the cap.

According to another variant, the means for holding the suture thread fitted on the needle is constituted by an annular protuberance formed inside the cap and defining a space for guiding the thread.

Advantageously, the means for holding the suture thread fitted on the needle is located in a transverse plane passing through the distal end of the friction zone of the needle.

According to a variant, the means for holding the suture thread fitted on the needle is located in a transverse plane passing through the proximal end of the friction zone of the needle.

Advantageously, the cap features an angular index.

According to an advantageous embodiment, the cap is made up of one or more functional elements that can be assembled together to form an embodiment of the present disclosure in its final form.

### BRIEF DESCRIPTION OF THE DRAWINGS

The present disclosure will be described in more detail with reference to non-limiting example embodiments specifying the aforementioned advantages and considerations. A more specific description of embodiments of the disclosure will be given below.

FIG. 1 shows a front view and a side view of a first embodiment of the present disclosure comprising a slot and a proximal groove.

FIG. 2 shows a front view and a side view of a second embodiment of the present disclosure comprising two slots and a proximal groove.

FIG. 3 shows a front view and a side view of a third embodiment of the present disclosure comprising two slots and a distal groove.

FIG. 4 shows a profile view of the suture thread holding means according to several embodiments of the present disclosure.

FIG. 5 shows a set of views illustrating the four stages of interaction between an embodiment of the present disclosure and a needle gripping tool.

### DETAILED DESCRIPTION

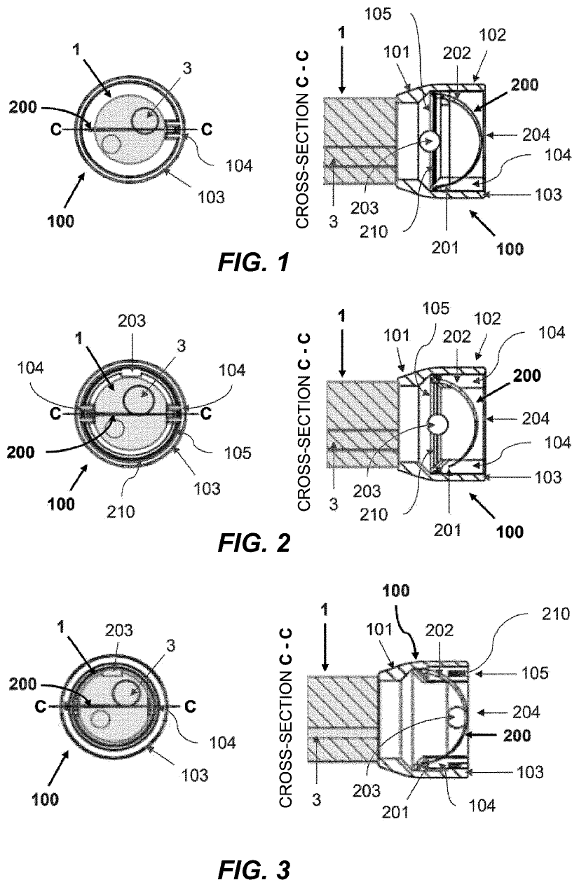

FIG. 1 shows a schematic front view from the distal end and a cross-sectional side profile view of an endoscopic cap (100) in the form of a hollow frustoconical tube having an opening at each end (101, 102). The proximal end (101) is made of a flexible plastic part (e.g., silicone or any other material with similar properties) enabling the cap (100) to be attached to the distal end of a flexible endoscope (1). The diameter and flexibility of this proximal end (101) enable it to be attached to a wide range of commercially available flexible endoscopes (1). The flexible endoscope (1) features a camera, an endoscopic lumen and an operating channel (3) for the passage of endoscopic tools, in particular, the needle gripping tool (300), notably the endoscopic needle holder.

The distal end (102) of the cap (100) consists of a tubular wall (103) made of round, transparent hard plastic (e.g., polycarbonate or any other material with similar properties). This tubular wall (103) features a temporary holding means (104) for a curved surgical needle (200). Depending on the size of the needle (200), the distal end (102) could be smaller than, the same size as or larger than the proximal end (101).

The tubular wall (103) has a housing constituting the temporary holding means (105) for keeping the suture thread (210) attached to the needle (200). The suture thread (210) advantageously features a plastic button (203) at its distal end for initial anchoring of the suture in the tissue to be sutured. Alternatively, the thread can also be held in place by adhesion.

The holding means (104) for the surgical needle (200), enabling the surgical needle (200) to be held within the cap (100) until it reaches the desired location in the gastrointestinal tract, is constituted in the example shown in FIG. 1 by a longitudinal slot formed on the inner surface of the tubular wall (103). The surgical needle (200) is then released into the gastrointestinal tract using the gripping tool (300).

The means (104) for holding the surgical needle (200) protects the walls of the gastrointestinal tract until it is extracted by the needle gripping tool (300). In the example shown in FIG. 1, it consists of a single slot (104) running the length of the tubular wall (103) of the cap (100), with the pointed end (201) of the needle (200) bearing against the inner surface of the tubular wall (103) of the cap (100), substantially in an area diametrically opposite the single slot (104).

The surgical needle (200) is positioned between the two edges of the longitudinal slot(s) to provide a grip strong enough, by dry friction, that it does not move on its own, simply due to the movement of the distal end of the endoscope (1), but weak enough to allow the needle gripping tool (300) to push the surgical needle (200) by axial movement, in order to deliver it.

According to the example described, the needle (200) has an arcuate shape extending over about 180°, and is made of a biocompatible material having an elasticity suitable for low deformation when the needle is introduced into the cap (100) of internal cross-section slightly smaller than the needle chord passing through the two zones (201, 202) that are furthest apart, generally the tip (201) and the opposite end (202) having the needle eye into which the suture thread (210) is pre-threaded.

The needle (200) can also have a straight shape, with both ends (201, 202) engaged in the longitudinal slot(s) (104).

### Alternative Embodiments

The means (104) for holding the surgical needle (200) can be made in forms other than a single slot (104) running along the length of the tubular wall (103) of the cap (100).

This can be achieved, as shown in FIG. 2, by two slots (104) each extending along two diametrically opposed longitudinal generatrices of the tubular wall (103) of the cap (100).

Irrespective of the design, the surgical needle (200) is arranged on the longitudinal axis of symmetry of the cap (100), giving it a central position relative to the distal end of the endoscope (1), so as not to significantly obstruct the camera's field of view and thus the endoscopist's visibility.

The position of the surgical needle (200) provides compatibility with different commercially available types of endoscopes (1). The cap (100) can be rotated relative to the endoscope (1) by a suitable angle so that the surgical needle is ideally positioned opposite the operating channel (3) of the endoscope (1).

Each of the first two embodiments can be further subdivided into two further embodiments wherein the longitudinal slot(s) (104) is/are arranged on only part of the inner surface of the tubular wall (103) of the distal end of the cap (100).

If the longitudinal slot(s) (104) is/are arranged over only part of the length of the distal end of the tubular wall (103), this will, in particular, provide more space for the placement of the holding means (105) for the suture thread (210).

The longitudinal slot(s) (104) of the holding means of the surgical needle (200) is/are raised from the surface of the tubular wall (103). In a first embodiment, the slot(s) (104) is/are represented by an addition of material with respect to the surface of the tubular wall (103) of the cap (100). In a second embodiment, the slot(s) (104) is/are formed by a removal of material with respect to the surface of the tubular wall (103).

The longitudinal slot(s) of the holding means (104) for the surgical needle (200) is/are open at its/their distal end to allow the release of the surgical needle (200) into the gastrointestinal tract using the needle gripping tool (300). This gripping tool passes through the operating channel (3) of the endoscope (1), then through the cap (100) in order to grasp the surgical needle (200) and remove it from its temporary holding means (104), pushing it toward the gastrointestinal lumen without letting go.

In another embodiment, the opening at the distal end of the slot (104) has a slope that allows the needle (200) to be withdrawn using the gripping tool (300), but also allows the needle (200) to be relocated in the opposite direction (with the needle tips (201, 202) toward the distal end (102) of the cap (100), that is, toward the lumen of the gastrointestinal tract). In this way, the tips (201, 202) of the needle (200) are held and protected by the slope so as not to injure the walls of the digestive tract when the system is removed.

According to another embodiment, the opening at the distal end of the slot(s) (104) is widened outwards—in the shape of a V—to enable the needle (200) to be directed toward the slot(s) (104) when it is replaced in the cap (100) at the end of the procedure.

FIG. 5 shows the four main stages of interaction of the needle gripping tool (300) with the cap (100) according to the present disclosure. In an initial phase, the endoscope (1) is placed in the patient's digestive system. The endoscopic cap (100) is placed at the distal end of the endoscope (1). The surgical needle (200) is placed in its holding means (104) in such a way that its ends (201, 202) are directed toward the proximal end (101) of the cap (100), toward the endoscope (1), and the inflection point (204) of its curvature is arranged toward the distal end (102) of the cap (100), toward the intestinal lumen. This position protects the walls of the gastrointestinal tract from injury or perforation, which could lead to complications for the patient. This position also makes it easier to grip the needle, as disclosed below. The needle gripping tool (300)—e.g., endoscopic needle holder—is arranged inside the operating channel (3) of the endoscope (1), retracted into a rear position.

In a second phase, the distal end of the needle gripping tool (300)—e.g., endoscopic needle holder—arranged inside the operating channel (3) of the endoscope (1) is exposed by axial advancement (translation) to the outlet of the operating channel (3) at the distal end of the endoscope (1). The needle gripping tool (300) is advanced until it comes into contact with the surgical needle (200). The position of the surgical needle (200) ensures an ideal distance between the outlet of the operating channel (3) at the distal end of the endoscope (1) and the curvature of the surgical needle, where the latter is caught. As a result, the gripping tool (300) for the surgical needle emerging from the operating channel (3) can protrude by a sufficient length to give it a degree of flexibility.

In a third step, this flexibility allows the surgical needle gripping tool (300) to deform slightly and thus to be deflected on contact with the surgical needle (200). The jaws of the surgical needle gripping tool are positioned laterally with respect to the needle. As deflection of the surgical needle gripping tool (300) causes stress on the latter, opening its jaws enables it to return to its initial position—releasing the stress—and thus to have the surgical needle (200) positioned between its jaws, in the area surrounding the inflection point (204). According to one variant, the holding means (104) for the needle (200) is formed by slots made of flexible material, enabling the surgical needle (200) to perform a slight lateral movement to facilitate the passage/deflection of the needle gripping tool (300). The needle gripping tool (300) is then actuated to close its jaws on the needle (200), enabling it to be firmly held and controlled. The forward axial translation (toward the intestinal lumen) of the gripping tool (300) thus enables the surgical needle (200) to be extracted from the longitudinal slot(s) (104) by pushing it toward the distal end (102) of the cap (100), toward the intestinal lumen, enabling the surgical needle (200) to be progressively delivered into the gastrointestinal tract without ever letting go. Since the needle gripping tool (300) can be rotated, the surgical needle (200) itself can be progressively and precisely controlled in rotation when held by the gripping tool (300).

### Suture Thread Housing

In one example, the holding means (105) for the suture thread (210) is provided by a groove-shaped housing in the inner face of the tubular wall (103) of the cap (100), so as not to increase its diameter. Indeed, the smaller the diameter, the easier it is to handle the cap (100) and to pass it through the gastrointestinal tract (particularly the esophagus).

The holding means for the suture thread (210) consists of one or more circular grooves (105). Alternatively, holding can also be achieved by adhesion, for example, with an adhesive that allows release when traction is exerted on the needle (200).

In a first embodiment, the circular groove(s) (105) is/are placed at the distal end (102) of the cap (100) near its opening, as shown in FIG. 3. They are located in a transverse plane at the distal end of the longitudinal slot(s) (104) for holding the surgical needle (200). In this way, the suture thread (210) emerging from one of the pointed ends (202) of the surgical needle (200) runs through the longitudinal slot(s) (104) holding the surgical needle (200) in place before winding into the circular groove(s) (105). With this embodiment, the length of the cap (100) is not increased, thus improving visibility for the endoscopist.

In another embodiment, the circular groove(s) (105) is/are located at the proximal end (101) of the tubular wall (103) of the cap (100); they are therefore located upstream of the longitudinal slot(s) (104).

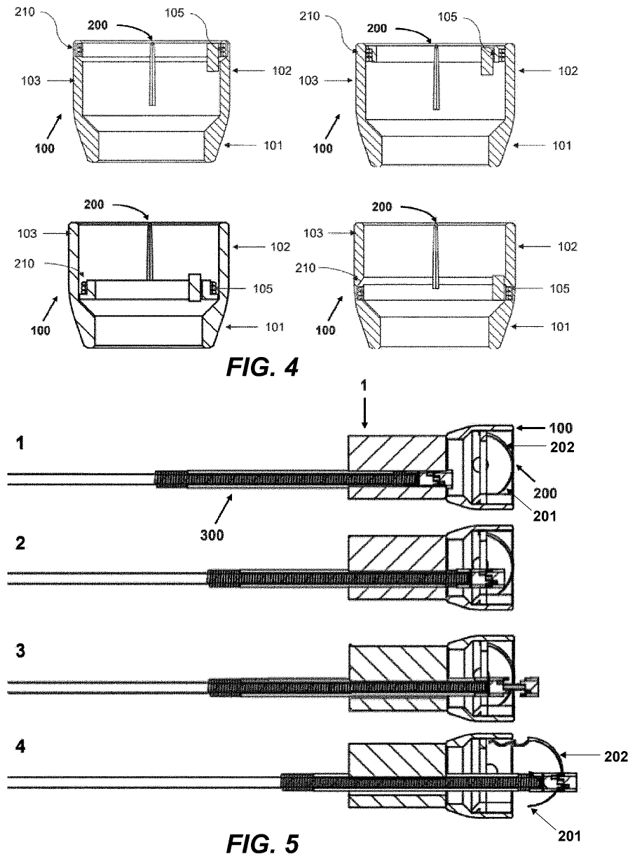

According to an embodiment shown in FIG. 4, the circular groove(s) (105) for holding the suture thread is/are raised with respect to the surface of the tubular wall (103) of the cap (100). In another embodiment, the circular groove(s) (105) is/are represented by a ring-shaped addition of material with respect to the surface of the tubular wall (103) of the cap (100).

In another embodiment shown in FIG. 4, the circular groove(s) (105) is/are represented by a material recess with respect to the surface of the tubular wall (103) of the cap (100) forming a narrow slit in the thickness of the tubular wall (103) of the cap (100). With this embodiment, the endoscopist's field of vision is not reduced, as no additional thickness is added to that of the tubular wall (103) of the cap (100).

The circular groove(s) (105) for holding the suture thread (210) is/are arranged around the entire circumference of the cap (100) parallel to the tubular wall (103) of the cap (100) so as to enable the entire length of the thread to be stored without obstructing the endoscopist's visibility or the passage of tools.

According to an alternative, the holding zones (105) of the suture thread (210) are not continuous but comprise a succession of holding segments.

The thread can be held in place by temporary adhesion, which does not withstand traction exerted by the operator, or by friction in the groove.

The circular groove(s) (105) of the suture thread holding means is/are open at its distal end to allow release of the suture thread into the gastrointestinal tract when the surgical needle (200) is withdrawn from its holding means (104) by the endoscopic needle holder (300).

The circular groove(s) (105) of the suture thread holding means can accommodate the suture thread (210) wound several times as well as a plastic button (203) arranged at its distal end. This plastic button (203) anchors the suture thread (210) at the start of the suture.

1. A surgical suture system, comprising: an endoscopic cap having a tubular wall, a needle, and a tool for gripping the needle, is the tool being axially movable relative to the cap, the tool being configured to be inserted into the lumen of an endoscope and to slide axially along an axis parallel to the longitudinal axis of the cap, wherein the needle is temporarily retained by means for holding the needle in the cap, when the gripping tool is in the rear position in a position suitable for gripping, and wherein the cap further has a means for holding a suture thread fitted on the needle.

2. The surgical suture system of claim 1, wherein the needle is arcuate in shape.

3. The surgical suture system of claim 1, wherein the holding means for a suture thread is formed by a housing.

4. The surgical suture system of claim 1, wherein the needle is held by a longitudinal slot provided on the tubular wall of the endoscopic cap.

5. The surgical suture system of claim 1, wherein the tubular wall of the cap has two diametrically opposed holding zones for the needle, at least one of the holding zones for the needle being constituted by two protruding slots defining a longitudinal guide slot for the ends of the needle.

6. The surgical suture system of claim 1, wherein a peripheral groove formed in the tubular wall of the cap constitutes the temporary holding means for the suture thread engaged on the needle.

7. The surgical suture system of claim 1, wherein an annular protuberance formed inside the cap and defining a space for guiding the thread constitutes the means for temporarily holding a suture thread engaged on the needle.

8. The surgical suture system of claim 1, wherein the means for temporarily holding the suture thread fitted on the needle is located in a transverse plane passing through the distal end of a friction zone of the needle.

9. The surgical suture system of claim 1, wherein the means for temporarily holding the suture thread fitted on the needle is located in a transverse plane passing through the proximal end of a friction zone of the needle.

10. The surgical suture system of claim 1, wherein the means for temporarily holding a suture thread is formed by temporary adhesion.

11. The surgical suture system of claim 1, wherein the cap features an angular index.

12. A surgical suture system, comprising:
an endoscopic cap configured to attach to a distal end of an endoscope, the cap having a tubular wall, the cap having diametrically opposed slots at the inner surface of the tubular wall, the slots configured to temporarily secure a needle therein, the cap having a peripheral groove extending around the inner surface of the tubular wall, the groove configured to temporarily retain a suture thread therein;
a needle temporarily retained in the slots of the cap; and
a suture thread fastened to the needle at one end thereof, the suture thread temporarily retained in the peripheral groove of the cap.

13. The surgical suture system of claim 12, wherein the needle is arcuate in shape.

14. The surgical suture system of claim 12, wherein the peripheral groove of the cap comprises a housing at the inner surface of the tubular wall.

15. The surgical suture system of claim 12, wherein the slots are recesses formed in the inner surface of the tubular wall.

16. The surgical suture system of claim 12, wherein the slots are defined by protrusions protruding inwardly from the inner surface of the tubular wall.

17. The surgical suture system of claim 12, wherein the peripheral groove is a recess formed in the inner surface of the tubular wall.

18. The surgical suture system of claim 12, wherein the peripheral groove is defined between protrusions protruding inwardly from the inner surface of the tubular wall.

19. The surgical suture system of claim 12, wherein the peripheral groove is located in a transverse plane passing through the distal end of the needle.

20. The surgical suture system of claim 12, wherein the peripheral groove is located in a transverse plane passing through the proximal end of a friction zone of the needle.
